(19) United States
(12) Patent Application Publication
(10) Pub. No.: US 2016/0051451 A1
(43) Pub. Date: Feb. 25, 2016

(54) HIGH-STRENGTH, TRANSLUCENT MG-HIGH QUARTZ MIXED CRYSTAL GLASS CERAMICS

(71) Applicant: Fraunhofer-Gesellschaft zur Foerderung der angewandten Forschung e.V., München (DE)

(72) Inventors: Bernhard Durschang, Rottendorf (DE); Joern Probst, Kuernach (DE); Axel Katzschmann, Schwetzingen (DE)

(21) Appl. No.: 14/781,510

(22) PCT Filed: Mar. 27, 2014
(86) PCT No.: PCT/EP2014/056202

§ 371 Date: Sep. 30, 2015

(30) Foreign Application Priority Data
Apr. 2, 2013 (DE) 10 2013 103 285.0
Jul. 31, 2013 (DE) 10 2013 108 216.5

(51) Int. Cl. A61K 6/033; C03C 4/00; C03B 32/02; A61K 6/027; A61K 6/02; C03C 10/00; C03C 4/12

(52) U.S. Cl. CPC A61K 6/033; C03C 10/0009; C03C 10/0045; C03C 4/0021; C03C 4/12; A61K 6/0273; A61K 6/0215; A61K 6/0235; A61K 6/024; C03B 32/02

(57) ABSTRACT

The invention relates to the use of glass ceramics based on a high quartz mixed crystal system for dental purposes, which can be easily mechanically processed in an intermediate stage of crystallization and present high-strength, highly translucent and chemically stable glass ceramics following complete crystallization, wherein said glass or ceramics still have phosphorus and a transition metal compound, selected from titanium and zirconium or a mixture thereof.

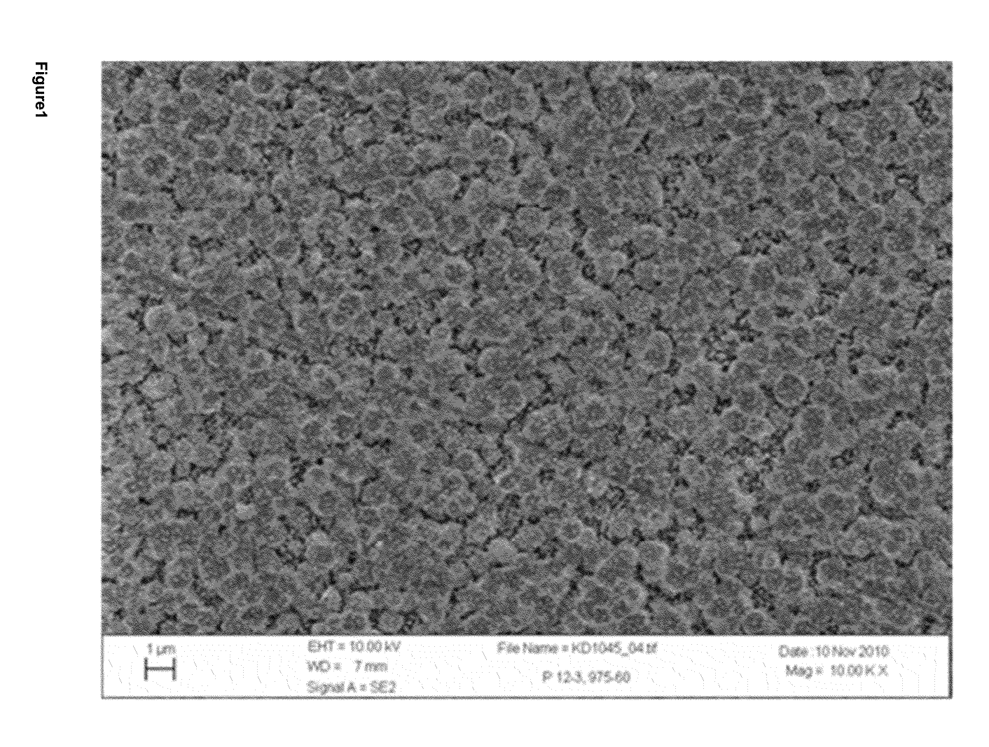

The invention relates to the use of glass ceramics based on a high quartz mixed crystal system for dental purposes, which can be easily mechanically processed in an intermediate stage of crystallization and present high-strength, highly translucent and chemically stable glass ceramics following complete crystallization.

High quartz mixed crystal glass ceramics are well known from literature; a number of patent publications relate to this glass ceramics system. Primary areas of application of the compositions mentioned therein are high-strength substrates for information recording media (e.g. substrates for magnetic storage disks), hotplates, mirror substrates, and fire protection materials (e.g. fireplace windows), as specified in EP 1 391 438 A1, DE 199 39 787 A1, DE 10 2004 024583 A1, and EP 1 029 830 A1.

All of these patents are based on a system consisting of the primary compounds: lithium oxide, aluminum oxide, and silicon oxide. It is distinguished in particular by its minimal and negative thermal expansion coefficients.

DE 20 2004 009 227 U1 suggests a glass/glass ceramic/metal embodiment for a light fixture having a glass or glass ceramic body, wherein said body may consist of Li₂O-Al₂O₃SiO₂ glass ceramics, which may contain MgO, TiO₂ and/or ZrO₂, and optionally P₂O₅ as well.

Glass ceramics are mentioned in DE 103 46 197 A1, which are suitable as a substrate for optical and electronic components and contain SiO₂, B₂O₃, Al₂O₃, MgO, TiO₂, ZrO₂, and optionally P₂O₅ as well.

In addition to lithium-high quartz mixed crystal glass ceramics, magnesium-high quartz mixed crystals are familiar as well. In this case, we should mention DE 26 02 429 C2, U.S. Pat. No. 6,953,756 B2, and U.S. 2008/0207425 A1. For dental applications, particularly for glass ceramics that can be processed with CAD/CAM, we should refer to the article “Dental Prostheses made of High-Strength Glass Ceramics” available online at: www.git-labor.de/forschung/materialien/zahnersatz-aus-hochfesten-glaskeramiken.

Glass ceramics capable of being processed with CAD/CAM are generally distinguished by the fact that the respective material can be readily processed in an intermediate stage with a minimal strength using CAM and subsequently converted into high-strength glass ceramics. Said glass ceramics are known from the lithium silicate system. In this case, e.g. DE-A-24 51 121 from 1974 demonstrates that glass ceramics containing lithium metal silicate as a primary phase have a reduced strength compared to glass ceramics containing lithium disilicate as the only crystalline phase. This principle was utilized to initially produce glass ceramics in a two-stage crystallization process, which is capable of being readily mechanically processed, e.g. by means of CAD/CAM procedures, and to subsequently process them in a second crystallization stage for dental glass ceramics. This method is suitable for being able to use dental restorations according to the so-called chairside method. With this method, an individually tailored crown/onlay/inlay is milled from a glass ceramic block in the dental practice after the first crystallization stage by means of CAD/CAM, which is subjected to a second crystallization stage in a special oven and applied to the patient directly during the initial and only dental visit—see, for example, DE 10 2005 028637 A1. With an addition of stabilizers, such as zirconium oxide, said glass ceramics were able to further improved with respect to strength and translucency—see WO 2012/059143.

However, it is preferable to provide glass ceramics for dental purposes, which not only have a high strength, but also such a structure that the development of fractures due to microstresses is prevented to the greatest extent possible. Such microstresses may occur, for example, as a result of the tempering steps necessary for the multi-stage production of glass ceramics.

Beginning with this consideration, the inventors set an additional objective that glass ceramics intended to have these preferable properties should have a minimal amount of lithium or be free of lithium, for lithium salts have been used for more than 60 years for the treatment of affective disorders, such as bipolar disorders or depressions. In this connection, the physiological effect of lithium salts is widely unknown as they influence countless processes in the human body. Lithium potentially reduces the probability of a further affective episode as it decreases a norepinephrine excess during manic episodes and activates the production of serotonin during depressive episodes. At the same time, the therapeutic index of lithium is low, i.e. even a merely slight increase of lithium ions in the body beyond the amount commonly used therapeutically may cause additional and hazardous side effects. Now, lithium cations are relatively readily soluble due to their size and, in a moist environment, could potentially escape from a compound with silicate and phosphate anions over the long term at least in very small quantities. Whether or not this actually occurs, and moreover whether or not this could be problematic in the case of dental applications, is unknown. However, it would therefore be beneficial if glass ceramics were available having a minimal amount of lithium ions or free of lithium ions.

Surprisingly, the inventors of the present invention were able to determine that the task at hand can be completed by providing glass ceramics of the system MgO-Al₂O₃-SiO₂, which on one hand only contains a minimal amount of lithium oxide or is free of lithium oxide, but on the other has significant amounts of phosphorus oxide. If glass is melted from this material and brought to a maximum temperature of 900° C. in one or two steps, a material containing crystals is achieved, which can still be readily processed, e.g. by means of CAD/CAM. If it is heated to more than 900° C. in a subsequent step, a high-strength material is produced, which is highly transparent and also has a better chemical resistance than known dental glass ceramics.

In the case of the following explanations, it is necessary to note that the information regarding mass is always specified for the respective cations in relation to pure oxide ceramics; if the glass ceramics contain other anions as well, appropriate corrections must be made. The information is provided in mass percent; accordingly mass percentages (MP) to one hundred parts of composition.

Glass ceramics from the system magnesium oxide/aluminum oxide/silicon dioxide (MAS) are known for their favorable mechanical properties, particularly their high strength. This system also serves the present invention as a starting system. In this regard, the three compounds are variable in broad ranges. In particular, a very small percentage of MgO of approx. 1 mass percent may already be sufficient; however this percentage can also increase up to 20 mass percent. The percentage of Al₂O₃ can fluctuate between approx. 12 and 30 mass percent; the percentage of SiO₂ can be at 30 to 60 mass percent. Most often, the percentage of MgO is at more than 5 mass percent; the percentage of Al₂O₃ is frequently not less than 15 mass percent. The percentage of SiO₂ is frequently in the range between 35 and 55 mass percent. Preferable ranges are: MgO percentage 6.0 to 14.5 mass percent; Al₂O₃ percentage 16 to 27 mass percent; SiO₂ percentage 40 to 55 mass percent.

It is still preferable that the ceramics contains little or no lithium. In the context of the invention, the percentage must be below 5 mass percent Li₂O; preferably it is below 2 mass percent, more preferably there are only traces (<1 mass percent to 0 mass percent) of Li₂O present, which were, e.g. unintentionally introduced.

In the state of the art, we know to add phosphorus to lithium silicate glass ceramics as a nucleating agent. It came as a complete surprise that the addition of phosphorus to the present, lithium-free or low-lithium MAS glass ceramics counteracts the propagation of microcracks if specific conditions for the hardening of the second stage are met, which will be explained in further detail below. Initially, said phosphorus crystallizes in the process in an individual phosphoric phase, which causes the formation of several relatively small MAS crystals that crystallize out as β-quartz. As a result, the number of crystals increases, which however remain relatively small (<2 μm) in the finally hardened glass ceramics. On one hand this produces a high strength, through which on the other hand, however, the development of cracks previously addressed above is conveniently inhibited. Furthermore, the small crystallites cause a high translucency.

The percentage of phosphorus, expressed in P₂O₅, should not be below 3.5 mass percent for this purpose. A range of 5 to 15 mass percent is preferable, more preferable is a range of 7.5 to 15 mass percent. High percentages are then particularly beneficial if the percentage of MgO is relatively low and vice versa.

The composition also contains substantial percentages of a transition metal oxide compound, chosen from TiO₂ and ZrO₂. Both of these substances should be included in a percentage of at least 5 mass percent, more preferably 8 mass percent, and may be present in a percentage of up to approx. 20 mass percent, preferably up to approx. 15 mass percent. In preferred configurations, either only TiO₂ or a mixture of TiO₂ and ZrO₂ is used. A percentage of at least 7.5 mass percent is beneficial, more preferably of at least 9.5 mass percent of TiO₂. ZrO₂ may be absent or likewise be present in these configurations.

Glass ceramics may contain, although this is not necessary, further additives, such as yttrium, lanthanum, cerium, germanium, tantalum and/or boric oxide in a quantity of up to approx. 15 mass percent.

The glass ceramics pursuant to the invention are preferably purely oxide ceramics. This means that they contain no other anions except O²⁻ ions, potentially with the exception of unintentionally introduced contaminants, which, however, preferably constitute no more than 1 mass percent with regard to the percentage of O²⁻ ions.

In preferred embodiments, they consist of the aforementioned compounds, with the exception of potentially unintentionally introduced cations. In particular, they should be free of additional alkali ions and/or alkaline earth ions. This limit for this is at a total of approx. 1 mass percent with regard to the respective oxides; however, these as well as additional cations are preferably only present in very minimal quantities (0.1 mass percent with regard to the oxides) or not at all.

Surprisingly, it was demonstrated that the glass compositions pursuant to the invention produce glass ceramics in the MAS high quartz mixed crystal system used, which are capable of being processed very well in a crystallization intermediate stage and have excellent strength properties, extraordinary translucency, and substantially increased chemical resistances in the final state. Although no clearly attributable phase transformations occur at this point, the material can be pre-crystallized in such a way that it can be converted with a brief thermal subsequent treatment from an intermediate stage capable of being readily processed by means of CAM to an extremely high-strength and translucent final material having an excellent chemical resistance.

For producing glass ceramics pursuant to the invention, a glass is initially melted from the selected compounds, which is normally achieved at 1500-1800° C. After being cooled to room temperature, a first crystallization phase occurs, which can take place in one or two stages. The temperatures for this are normally in the range of 750 to 880° C. depending on the individual material composition, although they can also reach 900° C. if the duration of the treatment, which is usually approx. 20 minutes to 4 hours, is kept relatively short. The product of this phase can be very readily processed.

The pre-crystallized material or product, which is potentially mechanically processed as desired, is then hardened in a second crystallization phase and, in the process, converted into high-strength glass ceramics. This hardening occurs at higher temperatures than those of the first crystallization phase—normally at approx. 925-1050° C.—and is normally shorter than the first crystallization phase as well. However, for this second hardening, it is necessary to note that the energy input via the temperature and time may not be too high—if the second and final hardening is conducted at temperatures, which are above a certain temperature difference compared to those of the first hardening, or if the duration of the second hardening is selected too long, an increased formation of cristobalite should be expected, through which the bending strength of subsequently hardened glass ceramics significantly decreases. This is due to the resulting easier propagation of cracks. A temperature that is between 40K and no more than 150K, preferably no more than 100K above the first hardening, should be selected in particular as the temperature of the second hardening, while the duration is normally only a fraction of that, which is used for the first hardening, and should be no more than half as long.

Based on the favorable ability to process said glass ceramics after the first tempering step (the first crystallization phase) and a very good hardness, crack resistance, and translucency after the subsequent hardening, glass ceramics pursuant to the invention are particularly well suited as dental materials, e.g. for dental restorations according to the chairside method described above. In this context, they serve as dental coloring in special configurations, for which common transition metal cations can be added, or they provide a slightly fluorescent effect similar to that of natural tooth fluorescence. This is achieved through the addition of cations of rare-earth elements, such as europium, terbium or praseodymium.

### DESIGN EXAMPLES

### Example 1

Glass was melted from the following compounds at approx. 1650° C. (specified in mass percentages (MP):

| | |
|---|---|
| SiO₂ | 41.5 |
| P₂O₅ | 8.0 |
| Al₂O₃ | 23.4 |
| MgO | 7.0 |
| TiO₂ | 5.7 |
| La₂O₃ | 12.6 |
| B₂O₃ | 1.8 |

After being cooled to room temperature, the material was subjected to an initial crystallization phase, namely in two stages—the first stage occurred at 780° C. for 3 hours and the second stage at 850° C. for 2 hours. This resulted in ceramics capable of being readily processed having a 3-point bending strength of 150 MPa.

They were subjected to a second crystallization phase (hardening phase) at 975° C. The crystallization was varied between 60 and 120 minutes. This resulted in glass ceramics having strength of 310 to 330 MPa (individual values >410 MPa), good translucency, and a low propagation of cracks.

As crystalline phases, high quartz mixed crystals (β-quartz structure) and lanthanum phosphate (LaPO₄) were able to be detected.

### Comparative Example 1

Example 1 was repeated, wherein the second crystallization phase (hardening phase) was conducted for 60 minutes at 1000 ° C. The strength was 160 MPa; cristobalite was detected in the Material as a crystallization phase.

### Example 2

As an additional example, glass similar to that in Example 1 was melted from the following compounds at approx. 1650° C. (specified in mass percentages (MP), wherein titanium oxide was partially replaced with zirconium oxide:

| | |
|---|---|
| SiO₂ | 40.7 |
| P₂O₅ | 7.8 |
| Al₂O₃ | 23.0 |
| MgO | 6.9 |
| TiO₂ | 1.9 |
| ZrO₂ | 5.7 |
| La₂O₃ | 12.3 |
| B₂O₃ | 1.8 |

After being cooled to room temperature, the material was subjected to various crystallization processes.

If the first crystallization was conducted for 2 hours at 900° C., this produced a still tolerable strength of 230 MPa for the processing phase. A subsequent treatment of 15 minutes at 960° C. produced a bending strength of 495 MPa (individual values >600 MPa).

As crystalline phases, high quartz mixed crystals (β-quartz structure) and lanthanum phosphate (LaPO₄) were able to be detected.

### Example 3

As an additional example, the following glass composition was melted:

| | |
|---|---|
| SiO₂ | 41.0 |
| P₂O₅ | 9.1 |
| Al₂O₃ | 23.1 |
| MgO | 7.0 |
| TiO₂ | 4.1 |
| ZrO₂ | 6.4 |
| La₂O₃ | 8.4 |
| B₂O₃ | 0.9 |

After being cooled to room temperature, the material was subjected to an initial crystallization phase of 930° C. for 2 hours and subsequently crystallized at 975° C. for 60 minutes in the second crystallization phase to the final properties. This resulted in glass ceramics having strengths of 445 MPa (individual values >550 MPa), good translucency, and a low propagation of cracks. As crystalline phases, high quartz mixed crystals (β-quartz structure), rutile (TiO₂), and lanthanum phosphate (LaPO₄) were detected. A higher degree of crystallization is revealed in the scanning electron microscope with crystals of a size of approx. 1 μm, see diagram 1.

### Example 4

Glass was melted from the following compounds at approx. 1650° C. (specified in mass percentages (MP):

| | |
|---|---|
| SiO₂ | 53.6 |
| P₂O₅ | 5.4 |
| Al₂O₃ | 17.9 |
| MgO | 13.6 |
| TiO₂ | 9.5 |

After being cooled to room temperature, the material was subjected to an initial crystallization phase, namely in two stages—the first stage occurred at 780° C. and the second stage at 850° C. The retention time was respectively between 20 minutes and 4 hours. This resulted in ceramics capable of being readily processed.

They were subjected to a second crystallization phase (hardening phase) at 950-1025° C. This was substantially shorter than the first crystallization phase and was between 10 and 80 minutes. This resulted in glass ceramics having a very favorable strength, good translucency, and favorable mechanical properties, particularly a high level of hardness and a low propagation of cracks.

### Example 5

Example 4 was repeated with the following glass composition:

| | |
|---|---|
| SiO₂ | 45.3 |
| P₂O₅ | 10.7 |
| Al₂O₃ | 25.6 |
| MgO | 7.7 |
| TiO₂ | 10.7. |

The results were comparable.

### Example 6

Example 4 was repeated with the following glass composition:

| | |
|---|---|
| SiO₂ | 41.0 |
| P₂O₅ | 9.1 |
| B₂O₃ | 0.9 |
| Al₂O₃ | 23.1 |
| MgO | 7.0 |
| TiO₂ | 4.1 |
| ZrO₂ | 6.3 |
| La₂O₃ | 8.4. |

The results were comparable.

What is claimed is:

1. Use of a magnesium oxide/aluminum oxide/silicon oxide glass or glass ceramics for application in dentistry, wherein said glass or glass ceramics have phosphorus and a transition metal compound, selected from the group consisting of titanium and zirconium or a mixture thereof.

2. Use of a magnesium oxide/aluminum oxide/silicon oxide glass or glass ceramics according to claim 1, wherein said magnesium oxide/aluminum oxide/silicon oxide glass or glass ceramics is a pure oxide glass.

3. Use of a magnesium oxide/aluminum oxide/silicon oxide glass or glass ceramics according to claim 1, having the following composition:

| | |
|---|---|
| SiO₂ | 30-60 MA |
| P₂O₅ | 3.5-15 MA |
| Al₂O₃ | 12-30 MA |
| MgO | 5-20 MA |
| Σ(TiO₂ + ZrO₂) | 5-20 MA |
| Σ MₓO_{y} | 0-15 MA, |

wherein M is selected from the group consisting of yttrium, lanthanum, cerium, germanium, tantalum, and boron.

4. Use of a magnesium oxide/aluminum oxide/silicon oxide glass or glass ceramics according to claim 3 having the following composition:

| | |
|---|---|
| SiO₂ | 35-55 MA |
| P₂O₅ | 5-15 MA |
| Al₂O₃ | 16-27 MA |
| MgO | 6-14.5 MA |
| Σ(TiO₂ + ZrO₂) | 5-15 MA |
| Σ MₓO_{y} | 0-12 MA. |

5. Use of a magnesium oxide/aluminum oxide/silicon oxide glass or glass ceramics according to claim 1, wherein the primary crystalline phase is a magnesium high quartz mixed crystal.

6. Use of a magnesium oxide/aluminum oxide/silicon oxide glass or glass ceramics according to claim 1, comprising a crystalline phase consisting of lanthanum phosphate.

7. Use according to claim 1 by means of CAD/CAM and/or for chairside applications.

8. Use according to claim 1, wherein said glass or glass ceramics have additional transition metal cations and are used as dental coloring.

9. Use according to claim 1, wherein a fluorescence additive is added to said glass or glass ceramics to produce a fluorescence effect emulating nature, preferably selected from cations of rare-earth elements, particularly preferably from Er³⁺, Eu³⁺, Tb³⁺, Pr³⁺, and Gd³⁺.

10. Use according to claim 1, comprising:
melting glass at temperatures between 1500 and 1800° C.,
active or passive cooling of said glass to room temperature,
causing a first crystallization by heating said glass to 750 to less than 900° C. for between 20 minutes and 4 hours, wherein glass ceramics capable of being readily processed are produced,
active or passive cooling of said glass ceramics obtained to room temperature,
causing a second crystallization by heating said glass to above 900° C. for a up to 120 minutes, during which the first crystallization is caused,
wherein said glass ceramics produced by the first crystallization are mechanically processed, wherein said mechanical processing stage comprises the production of a substitute usable in dentistry, preferably a crown, an onlay or an inlay, from a respective blank.

11. Use according to claim 10, wherein the second crystallization is conducted for no more than 80 minutes.

12. Use according to claim 10, wherein the first crystallization is conducted in two stages, wherein the first stage occurs at 750-800° C., preferably at approximately 780° C., and the second stage occurs at 820-880° C., preferably at approximately 850° C.

13. Use according to claim 10, wherein the second crystallization occurs at 950-1025° C., provided that the selected temperature is not more than 150K above that of the first crystallization.
